(19) United States
(12) Patent Application Publication
(10) Pub. No.: US 2017/0304433 A1
(43) Pub. Date: Oct. 26, 2017

(54) VACCINE COMPOSITIONS AND METHODS OF USE TO TREAT NEONATAL SUBJECTS

(71) Applicant: Wake Forest University Health Sciences, Winston-Salem, NC (US)

(72) Inventors: Martha A Miller, Clemmons, NC (US); Stephen Bruce King, Walnut Cove, NC (US)

(21) Appl. No.: 15/517,698

(22) PCT Filed: Oct. 7, 2015
(86) PCT No.: PCT/US15/54395

§ 371 Date: Apr. 7, 2017

Related U.S. Application Data
(60) Provisional application No. 62/062,005, filed on Oct. 9, 2014.

(51) Int. Cl. A61K 39/385; A61K 39/39; C12N 7/00; A61K 39/145; A61K 39/285; A61K 39/00

(52) U.S. Cl. CPC A61K 39/385; A61K 39/285; A61K 39/39; C12N 7/00; A61K 39/145; A61K 2039/572; A61K 2039/55516; A61K 2039/55; A61K 2039/5252; A61K 2039/575; A61K 2039/627; C12N 2710/24134; C12N 2760/16134; A61K 2039/55561

(57) ABSTRACT

The presently disclosed subject matter relates to the fields of pharmacology and medicine, and provides vaccine compositions and methods of use, particularly influenza compositions and methods of use to treat neonatal subjects.

### CROSS-REFERENCE TO RELATED APPLICATIONS

This application claims the benefit of U.S. Provisional Application No. 62/062,005, filed Oct. 9, 2014, which is incorporated herein by reference in its entirety.

### FIELD OF THE INVENTION

The invention relates to the fields of immunology and medicine, and provides vaccine compositions and methods of use, particularly influenza vaccine compositions and methods of use to treat neonatal subjects.

### BACKGROUND

There is clear need for the generation of vaccines that will be efficacious and safe in neonates. The neonatal immune system presents a number of challenges with regard to effective vaccination. This is, of course, due to the inability to perform studies in the most relevant population, the human neonate. Much of our current understanding of the neonatal immune response comes from studies in the mouse. A consistent finding in the mouse model is the propensity for differentiation of CD4⁺ T cells into Th2 cells (Adkins et al. (2004) Nat. Rev. Immunol. 4:553-564). There is support for this bias in humans based on studies using cord blood (Naderi et al. (2009) Clin. Exp. Med. 9:29-36). In addition, in human neonates there is also evidence for a generalized defect in responsiveness in T cells (Adkins et al. (2004) Nat. Rev. Immunol. 4:553-564; Winkler et al. (1999) J. Infect. Dis. 179:209-216; Xainli et al. (2002) J. Immunol. 169:3200-3207; Randolph (2005) NeoReviews 6:e454-e462; Zhao et al. (2002) Clin. Exp. Immunol. 129:302-308; Harris et al. (1992) Proc. Natl. Acad. Sci. U.S.A 89:10006-10010; Clerici et al. (1993) J. Clin. Invest 91:2829-2836; Miscia et al. (1999) J. Immunol. 163:2416-2424). An additional mediator of reduced responsiveness in neonates appears to be the presence of a heightened T_{reg} response (Fernandez et al. (2008) J. Immunol. 180:1556-1564). T_{reg} cells are known to decrease the number of virus-specific T cells generated following infection in a number of models (e.g. Chappert et al. (2010) Eur. J. Immunol. 40:339-350; Haeryfar et al. (2005) J. Immunol. 174:3344-3351). Thus, a preponderance of T_{reg} could result in the failure to generate a sufficient number of effector T cells.

Antibody responses are also significantly decreased in neonates, with these individuals demonstrating a defect in the production of high level, high affinity antibody (Adkins et al. (2004) Nat. Rev. Immunol. 4:553-564). In humans, antibody responses in young infants are largely IgM. IgG responses are generally weak for the first year of life (Randolph (2005) NeoReviews 6:e454-e462). For example, infants immunized with measles vaccine at 6 months of age have a weaker antibody response compared to infants immunized at 9-12 months (Gans et al. (2003) Vaccine 21:3398-3405). This effect was independent of the presence of detectable maternal antibody. Interestingly in one report, peripheral blood B cells from neonates studied in vitro appeared to have few defects with regard to activation (Tasker and Marshall-Clarke (2003) Clin. Exp. Immunol 134:409-419). Thus, whether B cell intrinsic defects are a predominant contributor to the decreased antibody response or whether it is the result of defects in the helper cell response remains an open question.

The presently disclosed subject matter is directed to vaccine compositions and methods of use, particularly influenza vaccine compositions and methods of use to treat neonatal subjects.

### SUMMARY

In some aspects, the presently disclosed subject matter provides a conjugated compound of formula (I):

Q-Z-V (I)

wherein: Q is a Toll-like receptor 7 (TLR7) agonist and/or a Toll-like receptor 8 (TLR8) agonist; Z is a linker; and V is a viral particle or virus-like particle.

In other aspects, within the conjugated compound of formula (I), Z is a linker comprising an amine reactive N-hydroxysuccinamide group and a thiol reactive maleimide group. In further aspects, the linker further comprises a moiety selected from the group consisting of a straight alkyl chain, a cyclohexane group, a polyethylene glycol group, and an aromatic ring. In further aspects, within the conjugated compound of formula (I), Z is a linker at least about 2.0 Å in length. In other aspects, within the conjugated compound of formula (I), Z is a linker selected from Table 1.

In further aspects, within the conjugated compound of formula (I), the TLR7 agonist and/or TLR8 agonist is an imidazoquinoline compound or derivative thereof. In a particular aspect, the TLR7 agonist and/or TLR8 agonist is an imidazoquinoline compound of formula (II):

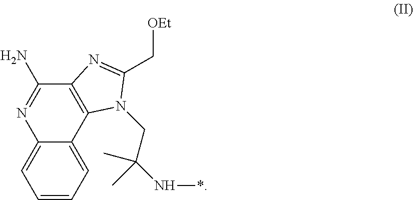

In other aspects, within the conjugated compound of formula (I), the viral particle or virus-like particle is derived from an enveloped virus, particularly wherein the enveloped virus is selected from the group consisting of an influenza virus, a vesicular stomatitis virus (VSV), an a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus. In a particular aspect, the enveloped virus is an influenza virus. In another particular aspect, the enveloped virus is a poxvirus, particularly a Vaccinia virus. In other aspects, the presently disclosed subject matter provides an immunogenic composition comprising any of the conjugated compounds disclosed herein.

In further aspects, the presently disclosed subject matter provides a method for inducing an immune response in a neonatal subject and/or a method of treating a viral infection in a neonatal subject, the method comprising contacting an immune cell within the neonatal subject with a conjugated compound of formula (I). In other aspects, the viral infection is caused by an enveloped virus selected from the group consisting of an influenza virus, a vesicular stomatitis virus (VSV), a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus. In a particular aspect, the enveloped virus is an influenza virus. In still further aspects, the conjugated compound is administered to the neonatal subject by a route selected from the group consisting of oral, nasal, sublingual, intravenous, subcutaneous, mucosal, ocular, respiratory, direct injection, and intradermally.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Drawings, which are not necessarily drawn to scale, and wherein:

FIG. 1 shows CRP levels in the plasma of infants twenty-four hours following vaccination. The presence of flagellin in the vaccine resulted in increased levels of CRP in vaccinated infants compared to the other vaccinated groups. Significance was determined by ANOVA. **p<0.01.

FIG. 2 shows anti-influenza IgG Ab in the plasma of African Green Monkey (AGM) infants post-primary and secondary immunization with IPR8-R848, IPR8+flagellin, or IPR8+m229 and in non-vaccinated control animals. R848 containing vaccines resulted in the highest induction of virus-specific antibody. *p<0.05, **p<0.002.

FIG. 3 shows anti-influenza IgG Ab in the plasma post-influenza virus challenge of AGM infants vaccinated with IPR8-R848, IPR8+flagellin, or IPR8+m229 and in non-vaccinated control animals. R848 containing vaccines resulted in the highest induction of virus-specific antibody. *p<0.05.

FIG. 4 shows ELISPOT analysis of IFNγ-producing cells in AGM infants vaccinated with IPR8-R848, IPR8+flagellin, or IPR8+m229 following influenza virus challenge. *p<0.05.

FIG. 5 shows virus load (FIG. 5A) and clearance (FIG. 5B), from the trachea and lung pathology on d14 post-challenge (FIG. 5C) in vaccinated AGM infants. Among neonatal animals challenged with influenza virus, those neonatal animals that were vaccinated with IPR8-R848 exhibited increased virus clearance from the trachea and lessened pathology compared to animals vaccinated with IPR8 in combination with other adjuvants. *p<0.05,**p<0.01.

FIG. 6 shows a schematic diagram of the synthesis of a conjugated compound comprising an agonist of Toll-like receptor 7 (TLR7)/Toll-like receptor 8 (TLR8), a linker of formula (II), and a viral particle or virus-like particle.

FIG. 7 shows a schematic diagram of the synthesis of a conjugated compound comprising an agonist of Toll-like receptor 7 (TLR7)/Toll-like receptor 8 (TLR8), a linker of formula (III), and a viral particle or virus-like particle.

FIG. 8 shows the ability to conjugate R848 to other viruses resulting in constructs with increased stimulatory capability. R848 can be effectively conjugated to other viruses to increase stimulatory capacity. IPR8-R848 is shown for comparison. IVV-R848 induced similar maturation as indicated by CD40 upregulation.

FIG. 9 shows a schematic diagram of the synthesized R848-SMCC linker (FIG. 9A) and its ability to promote maturation of RAW24 cells following conjugation to PR8 (FIG. 9B).

FIG. 10A shows the differential ability of linkers of various lengths to produce IPR8-R848 that is stimulatory for RAW264 cells. The addition of free (nonconjugated) R848 served as a positive control (FIG. 10B).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Respiratory and intestinal infections of infants between 1 and 6 months of life account for greater than 2 million deaths annually worldwide (Siegrist (2007) J. Comp. Pathol. 137 Suppl 1:S4-S9). Prominent among these infections is influenza virus, with one third of all infants infected in the first year of life (Glezen et al. (1997) Pediatr. Infect. Dis. J. 16:1065-1068). While wide-spread use of vaccines has been one of the greatest success stories in medicine, the vast majority of vaccines, including those for influenza, are not licensed for children <6 months of age, arguably a critical window for mortality and morbidity. This is in part a result of the large numbers of studies that suggest the neonatal immune system is compromised with regard to the ability to mount a protective immune response (Adkins et al. (2004) Nat. Rev. Immunol. 4:553-564; Zaghouani et al. (2009) Trends in Immunology 30:585-591).

The presently disclosed subject matter is directed to vaccine compositions and methods, particularly influenza vaccine compositions and methods of use to treat neonatal subjects.

### I. Conjugated Compounds and Immunogenic Compositions

In some aspects, the presently disclosed subject matter provides a conjugated compound of formula (I):
Q-Z-V (I)
   wherein: Q is a Toll-like receptor 7 (TLR7) agonist and/or a Toll-like receptor 8 (TLR8) agonist; Z is a linker; and V is a viral particle or virus-like particle.

A. Linkers

In other aspects, within the conjugated compound of formula (I), Z is a linker comprising an amine reactive N-hydroxysuccinamide group and a thiol reactive maleimide group. In further aspects, the linker further comprises a moiety selected from the group consisting of a straight alkyl chain, a cyclohexane group, a polyethylene glycol group, and an aromatic ring.

In other aspects, within the conjugated compound of formula (I), Z is a linker at least about 2.0 Å in length. In further aspects, within the conjugated compound of formula (I), Z is a linker at least about 2.1 Å, 2.2 Å, 2.3 Å, 2.4 Å, 2.5 Å, 2.6 Å, 2.7 Å, 2.8 Å, 2.9 Å, 3.0 Å, 3.5 Å, 4.0 Å, 4.5 Å, 5.0 Å, 5.5 Å, 6.0 Å, 6.5 Å, 7.0 Å, 7.5 Å, 8.0 Å, 8.5 Å, 9.0 Å, 9.5 Å, 10.0 Å, 10.5 Å, 11.0 Å, 11.5 Å, 12.0 Å, 12.5 Å, 13.0 Å, 13.5 Å, 14.0 Å, 14.5 Å, 15.0 Å, 15.5 Å, 16.0 Å, 16.5 Å, 17.0 Å, 17.5 Å, 18.0 Å, 18.5 Å, 19.0 Å, 19.5 Å, 20.0 Å, 21.5 Å, 22.0 Å, 22.5 Å, 23.0 Å, 23.5 Å, 24.0 Å, 24.5 Å, or 25.0 Å in length.

In other aspects, within the conjugated compound of formula (I), Z is a linker selected from Table 1.

TABLE 1
Linkers

| Name | Structure |
|---|---|
| SMCC (Succinimidyl 4- [N-maleimidomethyl] cyclohexane-1- carboxylate) | 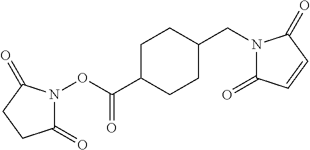 [SMILES: O=C(ON1C(=O)CCC1=O)C1CCC(CN2C(=O)C=CC2=O)CC1] |
| SM(PEG)ₙ (Succinimidyl- [N-maleimido- propionamido]-#- ethyleneglycol) ester) | 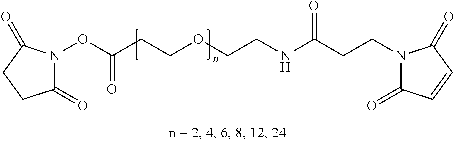 |
| AMAS (N[α-maleimido- acetoxy] succinimide ester) | 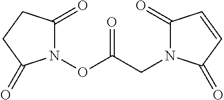 [SMILES: O=C(CN1C(=O)C=CC1=O)ON1C(=O)CCC1=O] |
| ML2-102 | 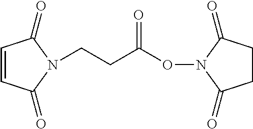 [SMILES: O=C(CCN1C(=O)C=CC1=O)ON1C(=O)CCC1=O] |
| ML2-103 | 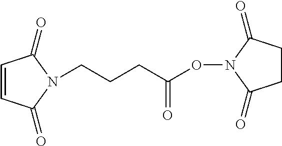 [SMILES: O=C(CCCN1C(=O)C=CC1=O)ON1C(=O)CCC1=O] |
| ML2-104 | 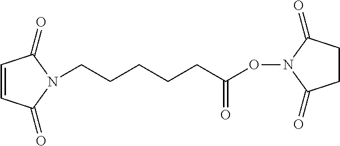 [SMILES: O=C(CCCCCN1C(=O)C=CC1=O)ON1C(=O)CCC1=O] |
| Mal-PEG2-NHS | 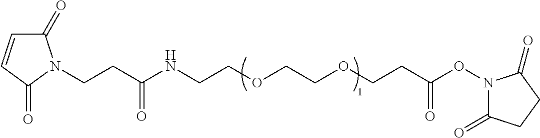 |
| Mal-PEG4-NHS | 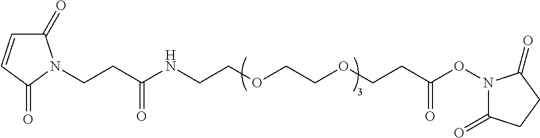 |
| Mal-PEG8-NHS | 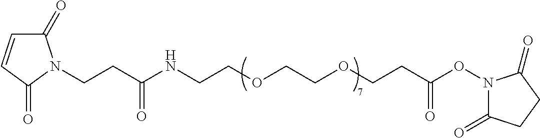 |
| Mal-PEG16-NHS | 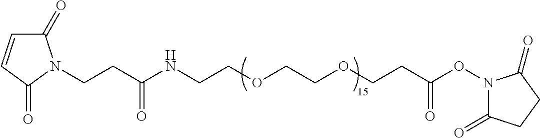 |

B. TLR7 Agonists and TLR8 Agonists

In certain aspects, the TLR7 agonist and/or TLR8 agonist within a conjugated compound of formula (I) is an imidazoquinoline compound or derivative thereof. In a particular aspect, the TLR7 agonist and/or TLR8 agonist is an imidazoquinoline compound of formula (II):

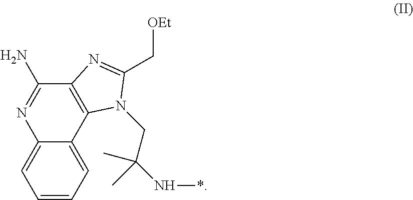

Toll-like receptors (TLRs) are expressed by cells of the immune system and by certain non-immune cells such as epithelial and tumor cells. To date, 10 TLR isoforms have been identified in humans. TLRs are type I membrane proteins with distinct cellular expression patterns and sub-cellular localization. TLR1, 2, 4, 5 and 6, are localized to the plasma membrane whereas TLR3, 4, 7, 8 and 9 reside in endosomal compartments, with TLR4 shuttling between the plasma membrane and endosomes. Engagement of TLRs with their specific ligands activates two major signaling pathways that are mediated by the adaptor proteins myeloid differentiation primary response gene 88 (MyD88) or TIR-domain-containing adaptor-inducing interferon-β(TRIF). Signaling cascades mediated by these pathways lead to the activation of transcription factors such as nuclear factor-kappa-B (NF-κB), activating protein-1 (AP-1) and interferon regulatory factors (IRFs) leading to the transcription of various genes for the production of inflammatory and anti-inflammatory cytokines, chemokines, and co-stimulatory molecules.

Since the induction of the adaptive immune system in vertebrates is heavily influenced by the innate immune system, TLR activation is an effective operation to effectively prime the adaptive response mediated by clonally distributed B- and T- cells. Several small molecule agonists of TLRs have been identified to shape adaptive immune responses to clear pathogens as well as to circumvent the process of carcinogenesis (Adams (2009) Immunotherapy 1: 949-964; Rakoff-Nahoum & Medzhitov (2009) Nature Reviews Cancer 9: 57-63).

TLR7 and TLR8 are, respectively, expressed in the endosomes of plasmacytoid dendritic cells (pDCs), macrophages and B cells, or myeloid dendritic cells (mDCs) and monocytes. TLR7 and TLR8 play a major role in the anti-viral response during viral infection by their ability to recognize single stranded RNA pathogen-associated molecular patterns (PAMPs). Several low molecular weight activators of TLR7 have been identified, which can be classified into three groups: imidazoquinolines, nucleoside analogs of purines, and 3-deazapurine derivatives (Hemmi et al. (2002) Nature Immunology 3:196-200; Lee et al. (2003) PNAS USA 100:6646-6651; Jones et al. (2011) Bioorganic Medicinal Chemistry Letters 21:5939-5943). Imidazoquinoline derivatives include 1H-imidazo[4,5-c]quinolones (U.S. Pat. No. 4,689,338) and imiquimod (3M-Aldara^{™, R-}837, S-26308). Other members of imidazoquinolines are Resiquimod (R-848, S-28609), Gardiquimod, and CL097 (InvivoGen), which in contrast to imiquimod are also ligands for the TLR8 receptor. Aldara™ is a cream formulation of imiquimod licensed for the topical treatment of anogenital warts, actinic keratosis and superficial basal cell carcinoma in humans. Nucleoside analogs of purines include 8-hydroxyadenines, such as 9-benzyl-8-hydroxy-2-(2-methoxyethoxy) adenine (SM-360320), (Kurimoto et al. (2004) Bioorganic Medicinal Chemistry 12:1091-1099) and the compound CL264 (InvivoGen), which is derived from SM-360320 by incorporating the amino-acid glycine, on the benzyl group. The third class of TLR7 agonists is 3-deazapurines, which are purine derivatives that include an amine functional group on the benzyl moiety (PCT Patent App. Pub. No. WO2007/093901).

TLR7 and TLR8 are targets for anti-cancer therapy (Smits et al. (2008) The Oncologist 13:859-875; Bourquin et al. (2011) Cancer Research 71:5123-5133; Hotz and Bourquin (2012) Oncoimmunology 1:227-228). A variety of different small molecule compounds that are TLR7 modulators, either purine or imidazoquinoline derivatives, have been reported for the treatment of infections and diseases, in particular to treat cancer of the skin and bladder, autoimmune diseases, allergic diseases and as adjuvants for vaccines (U.S. Patent App. Pub. No. 2011/0053893; U.S. Pat. No. 8,044,056; U.S. Pat. No. 7,485,432; U.S. Patent App. Pub. No. 2011/0070575; U.S. Patent App. Pub. No. 2011/0282061; U.S. Patent App. Pub. No. 2011/0229500; U.S. Patent App. Pub. No. 2010/0240623; U.S. Patent App. Pub. No. 2010/0210598).

C. Viral Particles and Virus-Like Particles

A “viral particle” is a generic term which includes a viral “shell”, “particle” or “coat”, including a protein “capsid”, a “lipid enveloped structure”, a “protein-nucleic acid capsid”, or a combination thereof (e.g., a lipid-protein envelope surrounding a protein-nucleic acid particle).

A “virus-like particle” refers to a small particle that contains one or more proteins from the outer coat of a virus.

In certain aspects, the viral particle or virus-like particle within a conjugated compound of formula (I) is derived from an enveloped virus, particularly wherein the enveloped virus is selected from the group consisting of an influenza virus, a vesicular stomatitis virus (VSV), a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus. In a particular aspect, the enveloped virus is an influenza virus. In another particular aspect, the enveloped virus is a poxvirus, particularly a Vaccinia virus. In other aspects, the presently disclosed subject matter provides an immunogenic composition comprising any of the conjugated compounds disclosed herein.

In some aspects, the synthesis of a conjugated compound of formula (I) comprises thiol reduction of the viral particle or virus-like particle (e.g., wherein the viral particle or virus-like particle is derived from an influenza virus). In other aspects, the synthesis of a conjugated compound of formula (I) comprises thiol reduction of the viral particle or virus-like particle (e.g., wherein the viral particle or virus-like particle is derived from a vaccinia virus).

D. Conjugation Methods

In some aspects, the synthesis of a conjugated compound of formula (I) comprises the conjugation of TLR7 and/or TLR8 agonist Q with linker Z to form construct Q-Z, followed by conjugation of construct Q-Z with viral particle V. In other aspects, construct Q-Z is synthesized as a single unit and does not require a separate conjugation step, thereby allowing for a single step conjugation of Q-Z with V to produce Q-Z-V of formula (I).

### II. Methods of Inducing an Immune Response in a Neonatal Subject

In other aspects, the presently disclosed subject matter provides a method for inducing an immune response in a neonatal subject and/or a method of treating a viral infection in a neonatal subject, the method comprising contacting an immune cell within the neonatal subject with a conjugated compound of formula (I) as described elsewhere herein. In certain aspects, the viral infection is caused by an enveloped virus selected from the group consisting of an influenza virus, a vesicular stomatitis virus (VSV), an a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus. In a particular aspect, the enveloped virus is an influenza virus. In another particular aspect, the enveloped virus is a poxvirus, particularly a Vaccinia virus. In still further aspects, the conjugated compound is administered to the neonatal subject by a route selected from the group consisting of oral, nasal, sublingual, intravenous, subcutaneous, mucosal, ocular, respiratory, direct injection, and intradermally. In other aspects, the subject is not limited to a neonatal subject, but may be selected from the group consisting of neonatal subjects, juvenile subjects, pregnant women, immunocompromised subjects, and elderly subjects.

The term “administering” as used herein refers to contacting at least a cell with an agent and/or polysaccharide antigens as defined herein. This term includes administration of the presently disclosed agents and/or polysaccharide antigens to a subject in which the cell is present, as well as introducing the presently disclosed agents into a medium in which a cell is cultured.

As used herein, the term “neonatal subject” refers to a newborn subject. Preferably the neonatal subject is a mammal in the first four weeks after birth. In other aspects, the neonatal subject is a mammal in the first two weeks after birth, or in the first week after birth.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments and the like).

In particular aspects, the subject is suffering from or susceptible to a disease, disorder, or condition associated with a viral infection caused by an enveloped virus. In some aspects, the enveloped virus is selected from the group consisting of an influenza virus, a vesicular stomatitis virus (VSV), an a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus. In a particular aspect, the enveloped virus is an influenza virus.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent and/or polysaccharide antigen can be administered prophylactically to prevent the onset of a disease, disorder, or condition, or to prevent the recurrence of a disease, disorder, or condition.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

In some embodiments, an effective amount of the conjugated compound of formula (I) is an amount that increases protective antibody levels and/or enhances a protective immune response in a subject in need thereof. In any of the above-described methods, administration of the conjugated compound of formula (I) as described herein results in at least about a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold increase in protective antibody levels and/or in a protective immune response in a subject in need thereof.

In any of the above-described methods, administration of a conjugated compound of formula (I) as described herein can result in at least about a 10% , 15%, 20%, 25%, 30%, 35%, 40%, 45%, 50%, 55%, 60%, 65%, 70%, 75%, 80%, 85%, 90%, 95%, or even 100% decrease in one or more (e.g., 1 , 2, 3, 4, 5, 6, 7, 8. 9, or 10) symptoms of a disease, disorder, or condition associated with a viral infection caused by an enveloped virus compared to a subject that is not administered a conjugated compound of formula (I) as described herein.

In any of the above-described methods, administration of a conjugated compound of formula (I) as described herein results in at least about a 10%, 15%, 20%, 25%, 30%, 35%, 40%, 45%, 50%, 55%, 60%, 65%, 70%, 75%, 80%, 85%, 90%, 95%, or even 100% decrease in the likelihood of developing a disease, disorder, or condition associated with a viral infection caused by an enveloped virus compared to a control population of subjects that are not administered a conjugated compound of formula (I) as described herein. Alternatively, in any of the above-described methods, administration of a conjugated compound of formula (I) as described herein results in at least about a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold decrease in the likelihood of developing a disease, disorder, or condition associated with a viral infection caused by an enveloped virus compared to a control population of subjects that are not administered a conjugated compound of formula (I) as described herein.

In some embodiments, the presently disclosed agents and/or polysaccharide antigens decrease, suppress, attenuate, diminish, arrest, or stabilize the development or progression of a disease, disorder, or condition, or the activity of a biological pathway, e.g., by at least 10%, 20%, 30%, 40%, 50%, 60%, 70%, 80%, 90%, 95%, 98%, 99%, or even 100% compared to an untreated control subject, cell, or biological pathway. Alternatively, in some embodiments, the presently disclosed agents and/or polysaccharide antigens decrease, suppress, attenuate, diminish, arrest, or stabilize the development or progression of a disease, disorder, or condition, or the activity of a biological pathway, e.g., by at least about a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold compared to an untreated control subject, cell, or biological pathway. By the term “decrease” is meant to inhibit, suppress, attenuate, diminish, arrest, or stabilize a symptom of a disease, disorder, or condition. It will be appreciated that, although not precluded, treating a disease, disorder or condition does not require that the disease, disorder, condition or symptoms associated therewith be completely eliminated.

In another aspect, the conjugated compound of formula (I) is administered to the neonatal subject by a route selected from the group consisting of oral, nasal, sublingual, intravenous, subcutaneous, mucosal, ocular, respiratory, direct injection, and intradermally.

Dosages of the therapeutic agents used in the presently disclosed subject matter must ultimately be set by an attending physician. Accordingly, the dosage range for administration will be adjusted by the physician as necessary. It will be appreciated that an amount of an agent required for achieving the desired biological response may be different from the amount of agent effective for another purpose. General outlines of the dosages are provided herein below.

Generally, a suitable dose of a conjugated compound of formula (I) as described herein for administration to a human neonatal subject will be in the range of from about 0.1 μg to about 100 μg per viral particle or virus-like particle; more particularly from about 0.1 μg to about 10 μg per viral particle or virus-like particle; alternatively from about 1 μg to about 50 μg per viral particle or virus-like particle; or from about 1 μg to about 25 μg per viral particle or virus-like particle; or from about 1 μg to about 15 μg per viral particle or virus-like particle; or from about 1 μg to about 10 μg per viral particle or virus-like particle; or from about 1 μg to about 5 μg per viral particle or virus-like particle. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

Generally, a suitable dose of a conjugated compound of formula (I) as described herein for administration to a human neonatal subject will be in the range of from about 0.1 mg/kg to about 500 mg/kg; alternatively, from about 1 mg to about 400 mg; preferably from about 1 mg to about 300 mg.

Actual dosage levels of the agents described herein can be varied so as to obtain an amount of the agent that is effective to achieve the desired therapeutic response for a particular subject, composition, route of administration, and disease, disorder, or condition without being toxic to the subject. The selected dosage level will depend on a variety of factors including the activity of the particular agent employed, or salt thereof, the route of administration, the time of administration, the rate of excretion of the particular agent being employed, the duration of the treatment, other drugs, agents and/or materials used in combination with the particular agent employed, the age, sex, weight, condition, general health and prior medical history of the patient being treated, and like factors well known in the medical arts.

### III. Definitions

A. Chemical Definitions

While the following terms in relation to conjugated compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(=O)O— is equivalent to —OC(=O)—; —OC(=O)NR— is equivalent to —NRC(=O)O—, and the like.

As used herein, where an internal substituent is flanked by bonds (for example, —NRC(O)—) the order of the atoms is fixed, the orientation of the group may not be reversed, and is inserted into a structure in the orientation presented. In other words —NRC(O)— is not the same as —C(O)NR—. As used herein the term C(O) (for example —NRC(O)—) is used to indicate a carbonyl (C=O) group, where the oxygen is bonded to the carbon by a double bond.

When the term “independently selected” is used, the substituents being referred to (e.g., R groups, such as groups R₁, R_{2,} and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R—substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxy, diethylamino, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

In certain embodiments, alkyl groups are C₁-C₆ alkyl groups or C₁-C₄ alkyl groups. The term “C₁-C₆ alkyl” as used herein means straight-chain, branched, or cyclic C₁-C₆ hydrocarbons which are completely saturated and hybrids thereof, such as (cycloalkyl)alkyl. Examples of C₁-C6 alkyl substituents include methyl (Me), ethyl (Et), propyl (including n-propyl (n-Pr, ⁿPr), iso-propyl (i-Pr, ¹Pr), and cyclopropyl (c-Pr, ⁰Pr)), butyl (including n-butyl (n-Bu, ⁿBu), iso-butyl (i-Bu, ¹Bu), sec-butyl (s-Bu, ^{s}Bu), tert-butyl (t-Bu, ¹Bu), or cyclobutyl (c-Bu, °⁰Bu)), and so forth.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of 0, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH_{3,} —CH₂—CH₂—NH—CH_{3,} —CH₂—CH₂—N(CH₃)—CH_{3,} —CH₂—S—CH₂—CH_{3,} —CH₂—CH₂₅—S(O)—CH_{3,} —CH₂—CH₂—S(O)₂—CH_{3,} —CH=CH—O—CH_{3,} —Si(CH₃)_{3,} —CH₂—CH=N—OCH_{3,} —CH=CH—N(CH₃)— CH_{3,}0—CH₃, -0—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

In the term “(cycloalkyl)alkyl”, cycloalkyl, and alkyl are as defined above, and the point of attachment is on the alkyl group. This term encompasses, but is not limited to, cyclopropylmethyl, cyclopentylmethyl, and cyclohexylmethyl. The alkyl group may be substituted or unsubstituted.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopropyl, cyclobutyl, cyclopentyl, cyclohexyl, and cycloheptyl.

Multicyclic cycloalkyl rings include adamantyl, octahydronaphthyl, decalin, camphor, camphane, and noradamantyl, and fused ring systems, such as dihydro- and tetrahydronaphthalene, and the like.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 141,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH=CH—CH=CH—; —CH=CH—CH₂—; —CH₂CH₂CH₂CH₂—, —CH₂CH=CHCH₂—, —CH₂CsCCH₂—, —CH₂CH₂CH(CH₂CH₂CH₃)CH₂—, —(CH₂)_{q}—N(R)—(CH₂),—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently.

The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, indazolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). The term “haloaryl,” however, as used herein, is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

As used herein, the term “alkylaryl” includes alkyl groups, as defined above, substituted by aryl groups, as defined above. The aryl group may be connected at any point on the alkyl group. The term C₄-C₁₆ alkylaryl includes alkylaryl groups having a total of 4 to 16 carbon atoms, counting the carbon atoms on the alkyl group and aryl group together. Examples of alkylaryl groups include but are not limited to benzyl (phenylmethyl), phenyl ethyl, and naphthylmethyl. The alkylaryl group may be substituted or unsubstituted. Substituents are not counted towards the total number of atoms in the alkylaryl group, so long as the total atoms in the substituent(s) are not larger than the alkylaryl group.

Further, a structure represented generally by the formula:

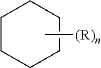

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

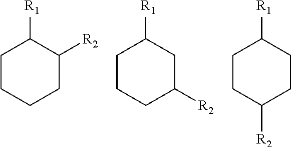

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

A substituent bearing a broken bond, such as the example shown below, means that the substituent is directly bonded to the molecule at the indicated position. No additional methylene (CH₂) groups are implied. The symbol ( [symbol] ) denotes the point of attachment of a moiety to the remainder of the molecule.

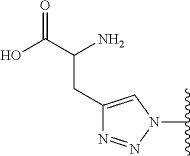 [SMILES: *n1cc(CC(N)C(=O)O)nn1]

Substituents bearing two broken bonds, such as the example shown below, means that the orientation of the atoms is as-indicated, left to right and should be inserted into a molecule in the orientation shown. No additional methylene (CH₂) groups are implied unless specifically indicated.

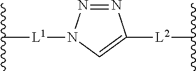

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, =O, =NR′, =N—OR′, —NR′R″, —SR′, -halogen, —SiR′R″R′″, —OC(O)R′, —C(O)R′, —CO₂R′,—C(O)NR′R″, —OC(O)NR′R″, —NR″C(O)R′, —NR′—C(O)NR″R′″, —NR″C(O)OR′, —NR—C(NR′R″)=NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH_{3,} —C(O)CF_{3,} —C(O)CH₂OCH_{3,} and the like).

Similar to the substituents described for alkyl groups above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)=NR″″, —NR—C(NR′R″)=NR′″ —S(O)R′, —S(O)₂R′, —S(O)₂NR′R″, —NRSO₂R′, —CN and —NO_{2,} —R′, —N_{3,} —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ,—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ᵣ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(=O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxyl, n-hexoxyl, and the like.

The term “alkoxyalkyl” as used herein refers to an alkyl-O—alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl. “Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl. “Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl—NH— group wherein acyl is as previously described. “Aroylamino” refers to an aroyl—NH— group wherein aroyl is as previously described.

The term “carbonyl” refers to the —(C=O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

The term ureido refers to a urea group of the formula —NH—CO—NH₂.

Unless otherwise explicitly defined, a “substituent group,” as used herein, includes a functional group selected from one or more of the following moieties, which are defined herein:

(A) —OH, —NH_{2,} —SH, —CN, —CF_{3,} —NO_{2,} oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH_{2,} —SH, —CN, —CF_{3,} —NO_{2,} halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH_{2,} —SH, —CN, —CF_{3,} —NO_{2,} halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH_{2,} —SH, —CN, —CF_{3,} —NO_{2,} halogen, unsubstituted alkyl, unsubstituted heteroalkyl, unsubstituted cycloalkyl, unsubstituted heterocycloalkyl, unsubstituted aryl, and unsubstituted heteroaryl.

A “lower substituent” or “lower substituent group,” as used herein means a group selected from all of the substituents described hereinabove for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

Throughout the specification and claims, a given chemical formula or name shall encompass all tautomers, congeners, and optical- and stereoisomers, as well as racemic mixtures where such isomers and mixtures exist.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C=N double bonds, and the like also can be present in the compounds described herein, and all such stable isomers are contemplated in the presently disclosed subject matter. Cis and trans geometric isomers of the compounds of the presently disclosed subject matter are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

The compounds herein described may have one or more charged atoms. For example, the compounds may be zwitterionic, but may be neutral overall. Other embodiments may have one or more charged groups, depending on the pH and other factors. In these embodiments, the compound may be associated with a suitable counter-ion. It is well known in the art how to prepare salts or exchange counter-ions. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Counter-ions may be changed, for example, by ion-exchange techniques such as ion-exchange chromatography. All zwitterions, salts and counter-ions are intended, unless the counter-ion or salt is specifically indicated. In certain embodiments, the salt or counter-ion may be pharmaceutically acceptable, for administration to a subject. Pharmaceutically acceptable salts are discussed later.

As used herein, a “protecting group” is a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting groups for protection of the carboxyl group, as described by Wutz et al. (pages 533-643), are used in certain embodiments. In some embodiments, the protecting group is removable by treatment with acid. Specific examples of protecting groups include but are not limited to, benzyl, p-methoxybenzyl (PMB), tertiary butyl (^{t}Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required and will be able to select an appropriate protecting group for use in a particular circumstance.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as pharmaceutically acceptable salts. The term “pharmaceutically acceptable salts” is meant to include salts of active compounds which are prepared with relatively nontoxic acids or bases, depending on the particular substituent moieties found on the compounds described herein. Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrates, (e.g. (+)-tartrates, (−)-tartrates or mixtures thereof including racemic mixtures), or teoclate. These salts may be prepared by methods known to those skilled in art. Other pharmaceutically acceptable salts may be found in, for example, Remington: The Science and Practice of Pharmacy (20^{th} ed.) Lippincott, Williams & Wilkins (2000).

Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

Also included are salts of amino acids such as arginate and the like, and salts of organic acids like glucuronic or galactunoric acids and the like, see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts. The neutral forms of the compounds may be regenerated by contacting the salt with a base or acid and isolating the parent compound in the conventional manner The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

B. General Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Example 1

There is clear need for the generation of vaccines that will be efficacious and safe in neonates. One of the limitations to forward movement in the area of vaccine optimization in infants is the inability to perform studies in the most relevant population, the human neonate. While investigators have often turned to the neonatal mouse model for such studies, we would argue that the more relevant model is the nonhuman primate (NHP). This is true with regard to both physiology and immune system development (Makori et al. (2003) Clin. Diagn. Lab Immunol. 10:140-153). Specifically: 1) mice are immunologically less mature at birth than are humans (Adkins et al. (2004) Nat. Rev. Immunol. 4:553-564); 2) the nonhuman primate lung is more similar in structure to the human than is the mouse lung (Irvin & Bates (2003) Respir.Res. 4:4); 3) there are stark differences in the composition of immune cells in peripheral blood of mice versus humans, with neutrophils comprising 50-70% of total cells in humans, while lymphocytes are the major cell type (70-90%) in mouse blood (Willems et al. (2009) Eur. J. Immunol. 39:26-35); and 4) the distribution and responsiveness of TLR receptors in the mouse and human differ (Willems et al. (2009) Eur. J. Immunol. 39:26-35), while there is a high degree of similarity between the NHP and humans (Ketloy et al. (2008) Vet. Immunol. Immunopathol. 125:18-30).

The cellular distribution of TLR is critical for studies that utilize agonists for TLR5, 7, and 8. Importantly, in contrast to NHP and human CD8+ T cells, mouse CD8+ T cells are not responsive to TLR5 agonists (Mizel & Bates (2010) J. Immunol. 185:5677-5682). Further, murine dendritic cells (DCs) exhibit limited maturation in response to flagellin (Means et al. (2003) J. Immunol. 170:5165-5175; Bates et al. (2009) J. Immunol. 182:7539-7547; Dearman et al. (2009) Immunology 126:475-484). With regard to TLR8, a number of studies demonstrate a lack of expression and/or function of this receptor in mice (Bauer et al. (2010) Proc. Natl. Acad. Sci. U.S.A. 107:E139; Hemmi et al. (2002) Nature Immunology 3:196-200; Jurk et al. (2002) Nat. Immunol. 3:499; Gorden et al. (2006) J. Immunol. 177:6584-6587; Luber et al. (2010) Immunity 32:279-289). This is significant as TLR8 agonists have been shown to suppress Treg cells, a critical target for the neonatal response as noted above. Finally, TLR7 is expressed on most DC and macrophages in mice, while it humans it is restricted to B cells and plasmacytoid DC (Iwasaki, A. and R. Medzhitov. 2004. Toll-like receptor control of the adaptive immune responses. Nat. Immunol 5:987-995). Thus the cells targeted by TLR7 agonists in mice and primates will differ substantially.

The present approach is innovative in that it exploits the non-human primate model for studies of neonatal immunity. The neonate has been only minimally utilized for to address these questions and never for the optimization of influenza vaccines in this high risk population.

This study tested the hypothesis that inclusion of the TLR agonists flagellin and R848 in an influenza virus vaccine would promote a robust protective immune response in neonates.

### Selection of Adjuvants

Flagellin. Flagellin (flg) is a TLR5 agonist that has proven to be a potent adjuvant for the induction of antibody responses in a number of experimental animal models (for review see Mizel & Bates (2010) J. Immunol. 185:5677-5682), including the Affrican Green Monkey (AGM) model utilized in our studies, as well as other nonhuman primate species (Honko et al. (2006) Infect. Immun. 74:1113-1120; Mizel et al. (2009) Clin. Vaccine Immunol. 16:21-28; Weimer et al. (2009) Vaccine 27:6762-6769). As a result of these studies, a flagellin adjuvanted vaccine against plague is currently entering clinical trials. Studies in mice demonstrating that flagellin inclusion in an inactivated influenza virus vaccine resulted in increased antibody titers and protection (Skountzou et al. (2010) Vaccine 28:4103-4112). Engagement of TLR5 provides a novel signal during vaccination as there is no known ligand for TLR5 in influenza virus.

The potency of flagellin as an adjuvant is in part due to its ability to induce activation of DC (Means et al. (2003) J. Immunol. 170:5165-5175). In addition, TLR5 agonists have the potential to act directly on primate T cells (Mizel & Bates (2010) J. Immunol. 185:5677-5682; Bates et al. (2009) J. Immunol. 182:7539-7547; Dearman et al. (2009) Immunology 126:475-484). Critically, there are data supporting the effectiveness of this adjuvant for the activation of T cells from neonates (McCarron & Reen (2009) J. Immunol. 182:55-62). Finally, flagellin effectively recruits T and B cells to secondary lymphoid sites, promoting more efficient activation of relevant immune effectors (Bates et al. (2008) Mech. Ageing Dev. 129:271-281; Honko & Mizel (2004) Infect. Immun. 72:6676-6679; Gewirtz et al. (2001) J. Immunol. 167:1882-1885). Thus, this adjuvant has the capacity to facilitate the generation of an immune response in neonates through its direct action on DC and T cells as well as its ability to induce proinflammatory cytokine/chemokine production and recruitment.

R848 (resiquimod). R848 is a ssRNA mimetic that has potent stimulatory capabilities for both TLR7 and TLR5. In experimental settings, R848 (or its closely related analog 3M-012) has been shown to increase cell mediated immune responses when incorporated into HBsAg (Ma et al. (2007) Biochem. Biophys. Res. Commun. 361:537-542) or HIV gag (Wille-Reece et al. (2005) Proc. Natl. Acad. Sci. U.S.A 102:15190-15194) vaccines. Although R848 can induce antibody production, it seems less efficient in this respect compared to other TLR agonists (Ma et al. (2007) Biochem. Biophys. Res. Commun. 361:537-542). While CD8+ T cell responses can be generated by co-administration of R848 and antigen, direct association of TLR7/8 adjuvants with a protein antigen, by either co-delivery in a water and oil adjuvant (Wille-Reece et al. (2006) J. Exp. Med. 203:1249-1258) or direct conjugation (Wille-Reece et al. (2005) Proc. Natl. Acad. Sci. U.S.A 102:15190-15194) results in a significantly increased CD8+ T cell response. Further, these adjuvants increase the quality of the CD8+ T cell response, i.e. a higher percentage of CD8+ antigen-specific cells exhibited polyfunctionality, i.e. production of IFNγ, IL-2, and TNFα (Wille-Reece et al. (2005) Proc. Natl. Acad. Sci. U.S.A 102:15190-15194). TLR8 agonists induce robust Th1 cytokines in vitro in neonatal APC (Levy et al. (2006) Blood 108:1284-1290). Further, TLR7/8 agonists have been shown to suppress Treg cells (Peng et al. (2005) Science 309:1380-1384).

Although inactivated influenza should contain ligands for TLR7 and TLR8, the failure to meet threshold level of TLR7/8 engagement may be responsible for the inability to induce a protective immune response in the setting of inactivated influenza virus. Further the virus associated ligand is likely only accessed by cells that take up virus, e.g. dendritic cells and macrophages as these are internal components. Delivery in the context of the vaccine allows efficient ligation on Treg.

### Effect of Flagellin or Conjugated R848 on the Ability of an Inactivated Influenza Vaccine to Induce an Immune Response and Provide Protection in Neonates

Vaccination. Prior to vaccination, mothers of the infants were screened to ensure that infants used in the study did not have circulating maternal derived influenza-specific IgG antibodies. Generally speaking, there is a 1:4 relationship in the age of monkeys versus humans (i.e., a 1 month old monkey is roughly equivalent to a 4 month old human). Accordingly, infants that were between 4 and 6 days old were used (approximating a 2-3 week old human infant). Infants were nursery reared.

The vaccine contained A/Puerto Rico/8/34/[H1N1]. Infants were vaccinated with 45 μg of inactivated PR8 virus (IPR8) conjugated to R848, IPR8+flagellin (flg) or with IPR8 plus 229 mutant flagellin (m229), which cannot activate TLR5, as a non-adjuvanted vaccine. As a control for vaccination, a group of animals received PBS. To prepare conjugated vaccine, an amine derivative of R848 was synthesized. R848 and SM(PEG)₄ in 100% DMSO were incubated together for 24 h. R848-SM(PEG)₄ was then incubated with reduced influenza virus, followed by dialysis to remove non-conjugated R848-SM(PEG)₄. The vaccine was then inactivated through treatment with 0.74% formaldehyde followed by dialysis. Successful conjugation was assessed by stimulation of RAW264 macrophage cells following incubation with similar amounts of conjugated versus non-conjugated vaccine. Preliminary studies showed that influenza alone did not appreciably activate the RAW264 cells, thus allowing assessment of conjugation.

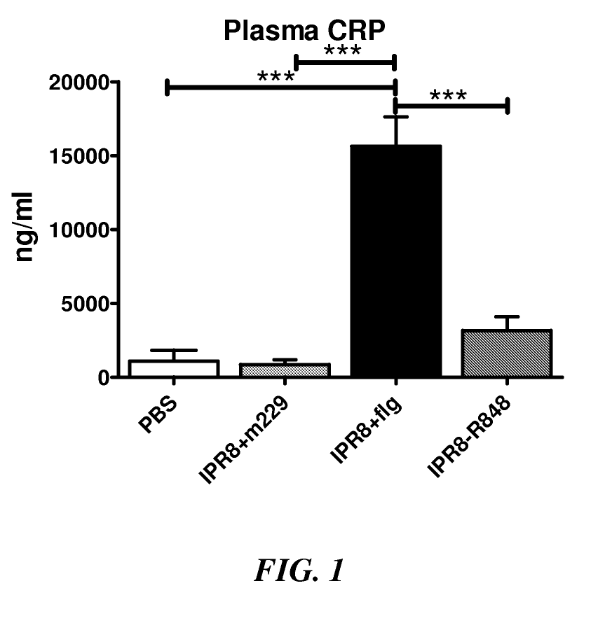

The present experiment included 4 neonate groups: unvaccinated (PBS), inactivated PR8 (IPR8)+m229, IPR8+flg, and the conjugate vaccine IPR8-R848. Vaccine was administered intramuscularly. Animals were boosted on day 21 post initial vaccination. Following vaccine administration animals were evaluated for the following: 1) visible inflammation at the site of injection; 2) changes in temperature for 48 hrs post-immunization; 3) C-reactive protein levels at 24 hrs post-immunization; 4) body weight; 5) food consumption, and 7) respiratory rate. No signs of adverse effects were apparent in the infants. Systemic CRP levels were upregulated in infants vaccinated with IPR8+flg. FIG. 1 shoes CRP levels in the plasma of infants 24 hours following vaccination. Animals receiving IPR8-R848 had minimal increases in CRP (FIG. 1).

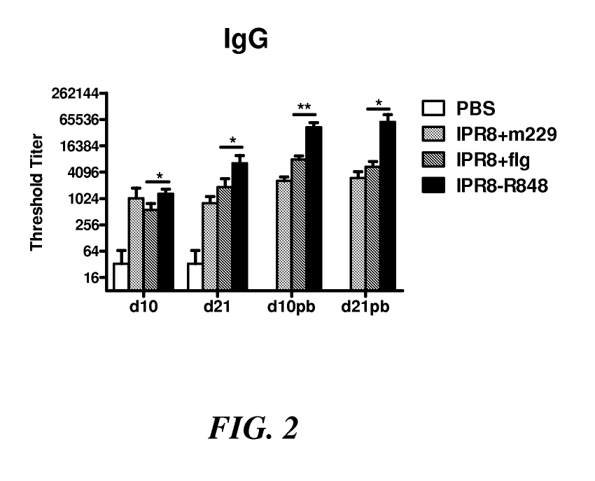

Influenza-specific IgG antibody response. FIG. 2 shows that immunization with the R848 conjugated vaccine resulted in a significantly increased level of influenza-specific IgG antibody compared to soluble flagellin conjugated vaccine (*p<0.05, **p<0.002). Circulating levels of influenza-specific IgG were measured 10 and 21 days following both prime and boost. Levels of anti-influenza virus IgG in vaccinated versus PBS treated infants were significantly increased in all cases (p<0.0001) at all times assessed. The presence of R848 resulted in a significant increase in virus-specific IgG at d21 post-primary vaccination as well as at d10 and 21 post-boost (pb) compared to animals that received m229. Although animals vaccinated with IPR8+flg had significantly increased influenza-specific antibody only at d10 post boost, the level of influenza virus specific IgG was further increased when R848 conjugated PR8 was used. These results show the presence of R848 conjugated to the virus particle has a robust adjuvant effect for elicitation of antibody in infants following vaccination and is superior to another candidate TLR agonist adjuvant, flagellin. Significance is indicated only for the comparison of flagellin and R848 conjugated vaccines for increased ease of viewing.

R848 promotes a greatly augmented systemic antibody response following virus challenge. An important goal of vaccination is to generate adaptive immune cells that can respond rapidly and effectively following pathogen challenge. To assess the recall potential of the immune response generated in the vaccinated infants, influenza virus (1×10¹⁰ EID₅₀) was delivered by the combined intratracheal and intranasal routes 24-26 days post boost.

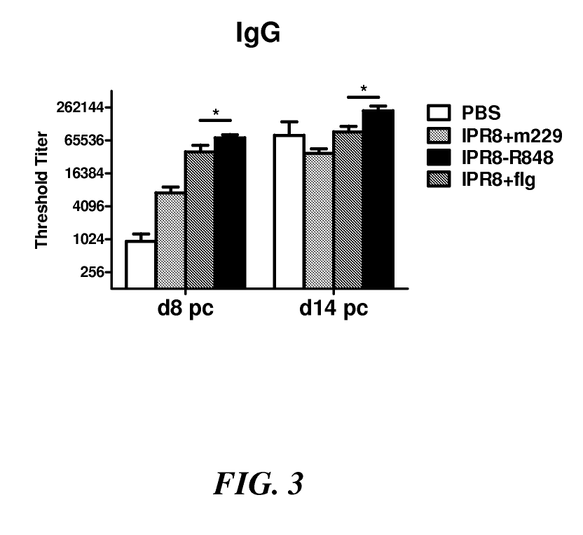

FIG. 3 shows that immunization with the R848 conjugated vaccine resulted in a significantly increased level of influenza-specific IgG antibody following virus challenge compared to soluble flagellin conjugated vaccine (*p<0.05). On day 8 and 14 post challenge (pc), the circulating level of virus-specific antibody was measured. At day 8 pc, animals that had received R848-conjugated vaccine exhibited greatly increased levels of virus specific IgG antibody compared to animals that received the negative control (m229) adjuvanted vaccine. As was observed following vaccination, the recall response in IPR8-R848 vaccinated infants was superior to infants vaccinated with IPR8+flg.

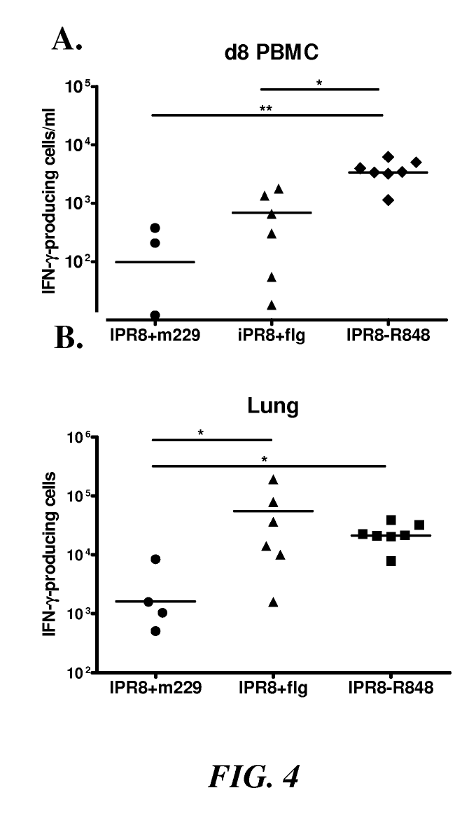

R848 adjuvanted vaccine results in an increased T cell response following challenge compared to flagellin adjuvanted and non adjuvanted vaccines. We next evaluated the T cell response in vaccinated animals following challenge. FIG. 4 shows ELISPOT analysis of IFNγ-producing cells in AGM infants vaccinated with IPR8-R848, IPR8+flagellin, or IPR8 following influenza virus challenge (*p<0.05). IFNγ producing cells were assessed at d8 pc in the blood and at d14 in the lung, TBLN, and spleen. Infants that received either of the R848-conjugated vaccines showed significantly enhanced numbers of IFNγ producing cells in the blood at d8 pc and in all tissues at d14 pc. Thus conjugation of R848 to inactivated influenza virus results in a robust IFNγ-producing recall response following virus challenge and this response is superior to flagellin.

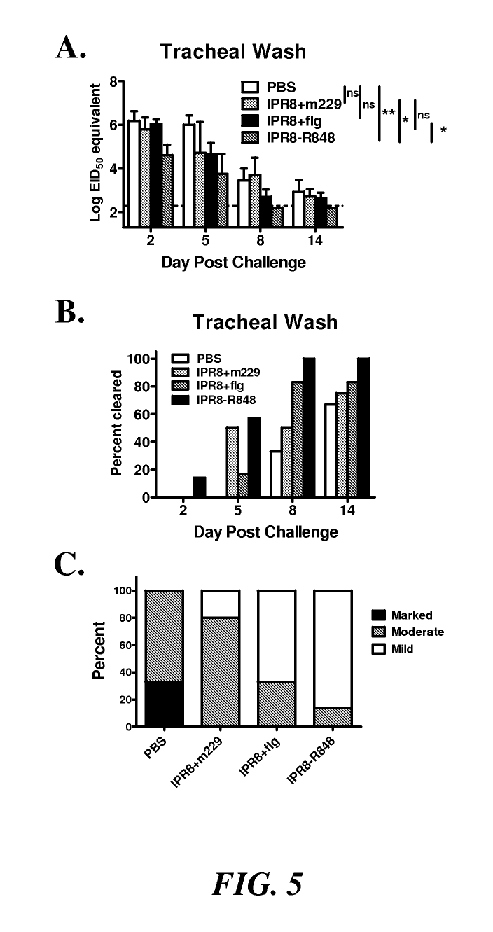

R848 is superior to flagellin adjuvanted and non-adjuvanted IPR8 with regard to viral clearance and protection from lung pathology following challenge. A protective vaccine should result in decreased viral burden and reduction in disease following challenge. The protective capacity of the candidate vaccines was determined by measuring viral load in the trachea over time by qRT-PCR. FIG. 5 shows virus load (FIG. SA) and clearance (FIG. 5B), from the trachea and lung pathology on d14 post-challenge (FIG. SC) in vaccinated AGM infants (*p<0.05,**p<0.01).

Infants vaccinated with IPR8-R848, but not IPR8+flg or IPR8+m229 exhibited a significantly decreased viral load in the trachea (FIG. SA) compared to the other constructs (FIG. 5B). Infants vaccinated with IPR8-R848 also cleared virus earlier than infants vaccinated with the other constructs.

We also assessed pathology in the lungs of vaccinated infants at 14 days post challenge. The slides were examined by light microscopy by an American College of Veterinary Pathologists board certified veterinary pathologist in a blinded fashion and evaluated for degree of inflammation and injury. Pathology assessment was based on interstitial and alveolar inflammatory cell infiltration and edema, pneumocyte hyperplasia, and bronchial degeneration and necrosis. Three of the four infants vaccinated with IPR8+m229 had moderate disease following challenge compared to 2 of 6 IPR8+flg vaccinated infants and 1 of 7 IPR8-R848 vaccinated infants (FIG. 5C).

### Example 2

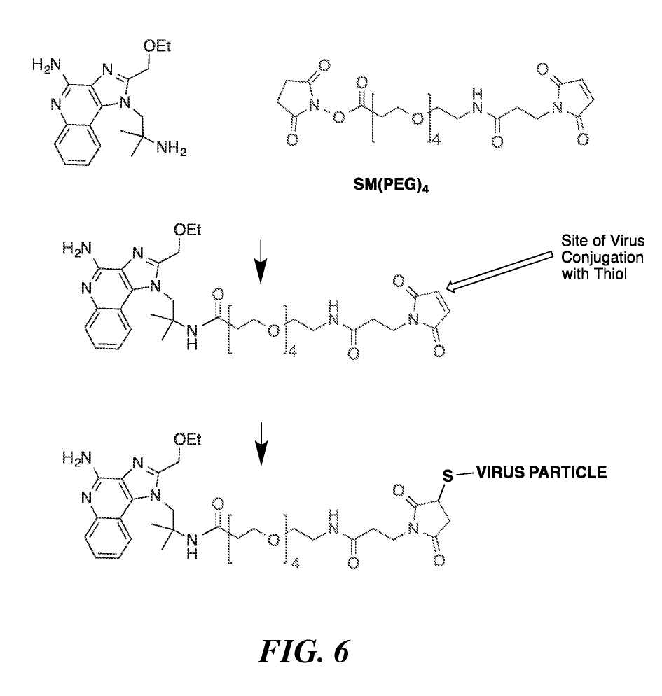

FIG. 6 shows a schematic diagram of the synthesis of a conjugated compound comprising an agonist of Toll-like receptor 7 (TLR7)/Toll-like receptor 8 (TLR8), a linker, and a viral particle or virus-like particle, wherein the linker is SM(PEG)₄ (Succinimidyl-[N-maleimido-propionamido]-4-ethyleneglycol) ester).

### Example 3

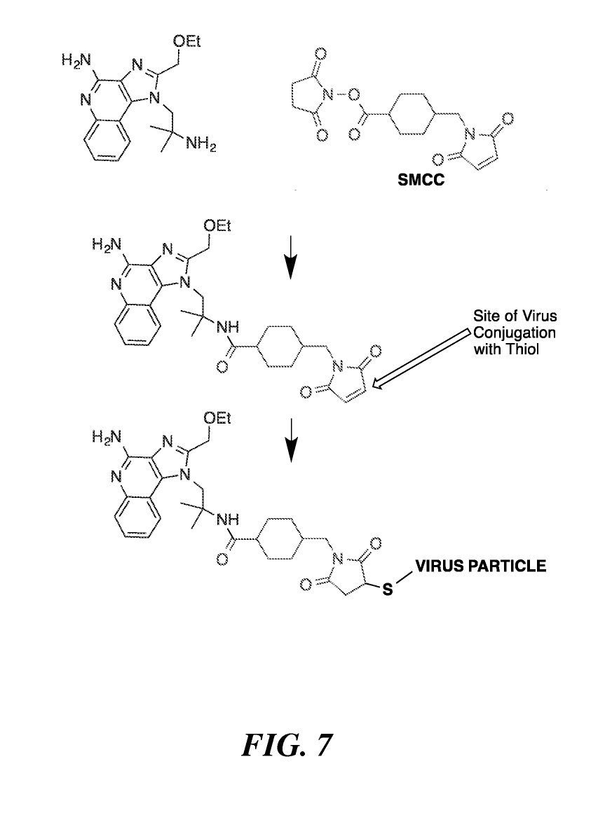

FIG. 7 shows a schematic diagram of the synthesis of a conjugated compound comprising an agonist of Toll-like receptor 7 (TLR7)/Toll-like receptor 8 (TLR8), a linker, and a viral particle or virus-like particle, wherein the linker is SMCC (Succinimidyl 4-[N-maleimidomethyl]cyclohexane-1-carboxylate).

### Example 4

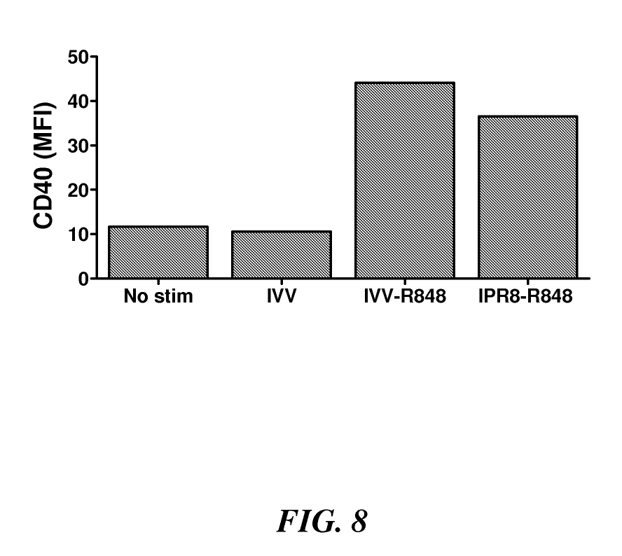

R848 can be conjugated to other viruses to promote maturation. We reasoned that the technology developed for conjugation of R848 to influenza virus should be readily applicable to other viruses. Vaccinia virus (VV) is an enveloped virus of the orthopox family. We used a similar approach for conjugating R848 with the exception of the omission of the reduction step. Analyses revealed abundant free thiols on vaccinia virus. Thus the requirement for reduction is virus dependent. FIG. 8 shows that that R848 can be effectively conjugated to other viruses to increase stimulatory capacity. IPR8-R848 is shown for comparison. IVV-R848 induced similar maturation as indicated by CD40 upregulation. The data in FIG. 8 show that R848-SM(PEFG)4-VV has greatly increased stimulatory capability compared to VV alone. Thus this approach can be broadly applied to viruses.

### Example 5

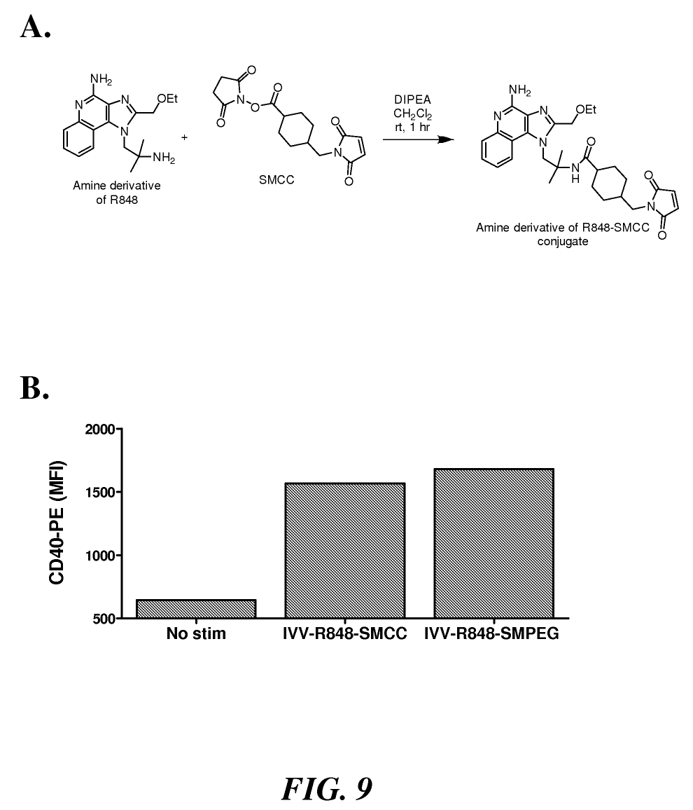

The amine derivative R848-SMCC linker can be synthesized as a single entity for single step conjugation to PR8. Linkage of R848 to virus would be facilitated by the ability to conjugate in one step through synthesis of R848 and linker as a single unit. To this end, R848 was synthesized with the linker SMCC (8.3 Å) using the following method. The amine derivative of R848 (1 equivalent) and diisopropyl ethyl amine (DIPEA, 1 equivalent) were added to a solution of succinimidyl 4-[N-maleimidomethyl]cyclohexane-1-carboxylate (SMCC, 1 equivalent) in CH₂Cl_{2.} After stirring this solution at room temperature for one hour, the solution was diluted with CH₂Cl₂, washed with 1N HCl and brine. The organic layer was dried over MgSO₄ and concentrated under vacuum to give a solid that was purified by silica gel flash chromatography whose structure was confirmed by ¹H, ¹³C NMR and mass spectrometry. A schematic of R848-SMCC synthesis is shown in FIG. 9A. R848-SMCC can be directly conjugated to PR8 and is stimulatory for activation of RAW264 macrophage cells (FIG. 9B).

### Example 6

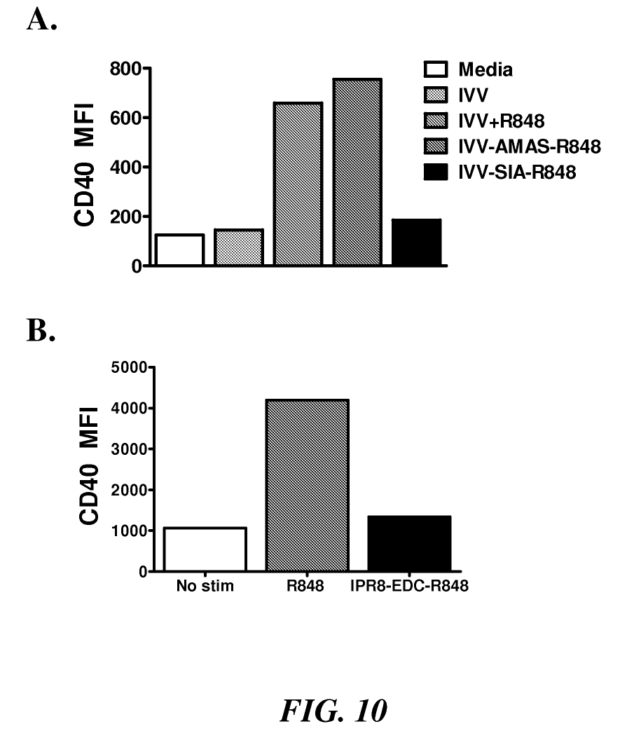

Linker requirement for conjugation to PR8 in a way that results in a stimulatory vaccine. To determine the breadth of linkers that can be used to conjugate the amine derivative of R848 to PR8, additional linkers of various lengths were utilized: EDC (1-ethyl-3-(3-dimethylaminopropyl)carbodiimide hydrochloride) (0 Å), SIA (succinimidyl iodoacetate) (1.5 Å), and AMAS (4.4 Å). The previously utilized linkers were 24.6 Å (SM(PEG)₄) and 8.3 Å(SMCC). The R848-linker-PR8 constructs were tested for the ability to induce maturation of RAW264 cells. FIG. 10A shows the ability of linkers of various lengths to produce an IPR8-R848 construct that is stimulatory for RAW264 cells. The addition of free (nonconjugated) R848 served as a positive control (FIG. 10B). AMAS, SMCC, and SM(PEG)₄ conjugated constructs were all effective for RAW264 maturation, whereas SIA and EDC linkers did not induce maturation (FIG. 10A).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. A conjugated compound of formula (I):
Q-Z-V (I)
wherein:
Q is a Toll-like receptor 7 (TLR7) agonist and/or a Toll-like receptor 8 (TLR8) agonist;
Z is a linker; and
V is a viral particle or virus-like particle.

2. The conjugated compound of claim 1, wherein Z is a linker comprising an amine reactive N-hydroxysuccinarnide group and a thiol reactive maleimide group.

3. The conjugated compound of claim 2, wherein the linker further comprises a moiety selected from the group consisting of a straight alkyl chain, a cyclohexane group, a polyethylene glycol group, and an aromatic ring.

4. The conjugated compound of claim 1, wherein Z is a linker selected from Table 1.

5. The conjugated compound of claim 1, wherein the TLR7 agonist and/or TLR8 agonist is an imidazoquinoline compound or derivative thereof.

6. The conjugated compound of claim 5, wherein the TLR7 agonist and/or TLR8 agonist is an imidazoquinoline compound of formula (II):

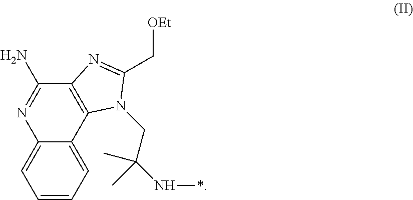

7. The conjugated compound of claim 1, wherein the viral particle or virus-like particle is derived from an enveloped virus.

8. The conjugated compound of claim 7, wherein the enveloped virus is selected from the group consisting of an influenza virus, a vesicular stomatitis virus (VSV), an a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus.

9. The conjugated compound of claim 8, wherein the enveloped virus is an influenza virus.

10. The conjugated compound of claim 8, wherein the enveloped virus is a poxvirus.

11. The conjugated compound of claim 10, wherein the poxvirus is a Vaccinia virus.

12. An immunogenic composition comprising the conjugated compound of claim 1.

13. A method for inducing an immune response in a neonatal subject, the method comprising contacting an immune cell within the neonatal subject with the conjugated compound of claim 1.

14. The method of claim 13, wherein the conjugated compound is administered to the neonatal subject by a route selected from the group consisting of oral, nasal, sublingual, intravenous, subcutaneous, mucosal, ocular, respiratory, direct injection, and intradermally.

15. A method of treating a viral infection in a neonatal subject, the method comprising contacting an immune cell within the neonatal subject with the conjugated compound of claim 1.

16. The method of claim 15, wherein the viral infection is caused by an enveloped virus.

17. The method of claim 16, wherein the enveloped virus is selected from the group consisting of an influenza virus, a vesicular stomatitis virus (VSV), an a human immunodeficiency virus (HIV), a herpesvirus, a papillomavirus, a poxvirus, a hepadnavirus, a flavivirus, a togavirus, a coronavirus, a hepatitis virus, an orthomyxovirus, a paramyxovirus, a rhabdovirus, a bunyavirus, and a filovirus.

18. The method of claim 17, wherein the enveloped virus is an influenza virus.

19. The method of claim 17, wherein the enveloped virus is a poxvirus.

20. The method of claim 19, wherein the poxvirus is a Vaccinia virus.

21. The method of claim 15, wherein the conjugated compound is administered to the neonatal subject by a route selected from the group consisting of oral, nasal, sublingual, intravenous, subcutaneous, mucosal, ocular, respiratory, direct injection, and intradermally.
